(19) United States
(12) Patent Application Publication
(10) Pub. No.: US 2012/0027827 A1
(43) Pub. Date: Feb. 2, 2012

(54) Method and Composition for Reducing Exhaled Carbon Dioxide Emissions

(76) Inventors: Michael Lefenfeld, New York City, NY (US); Tuhin Ganguly, Washington, DC (US)

(21) Appl. No.: 13/192,163

(22) Filed: Jul. 27, 2011

Related U.S. Application Data
(60) Provisional application No. 61/368,147, filed on Jul. 27, 2010.

(51) Int. Cl. A61K 9/68; A61Q 11/00; A61K 8/00

(52) U.S. Cl. 424/401; 424/48; 424/49

(57) ABSTRACT

A method and composition for reducing the amount of carbon dioxide exhaled by a user by placing a composition comprising a delivery vehicle and a carbon dioxide sequestering agent into a user's mouth and allowing the carbon dioxide sequestering agent to react with the carbon dioxide present in the user's breath thereby reducing the amount of carbon dioxide exhaled by the user.

The following patent application claims priority to U.S. Provisional Patent Application No. 61/368,147, filed on Jul. 27, 2010, which is hereby fully incorporated by reference for all purposes.

### FIELD OF INVENTION

The present invention relates generally to a method and composition which reduces the amount of carbon dioxide exhaled by an individual, and specifically to a composition comprising a carbon dioxide sequestering agent which captures carbon dioxide in a user's breath.

### BACKGROUND OF THE INVENTION

Over the last decade, the “green movement” has grown exponentially and is now influencing the daily behaviors of individuals, the practices and products of multinational companies, and the legislation passed by state and federal governments. One of the central initiatives of the movement is to reduce an individual's own “carbon footprint.” An individual can reduce their carbon footprint through practices which reduce their consumption of materials made of carbon-based products, like gasoline and plastics, or by reducing their own production of carbon dioxide, Reducing carbon dioxide emissions is beneficial because carbon dioxide, which is classified as one of the most common greenhouse gases, directly contributes to global warming. Carbon dioxide emissions are often reduced by consuming less gasoline, using alternative fuels, using energy efficient products, and producing less waste. These practices, however, do not address the largest source of carbon dioxide emissions: the world's growing population.

The majority of carbon dioxide emissions result from exhalation by people. Therefore, it would be advantageous to devise a method to reduce or sequester those direct repetitive emissions. Designing a method to reduce carbon dioxide from exhalation poses significant problems because, to be effective, the method cannot be intrusive, uncomfortable, or inconvenient by changing the normal human pathology. Accordingly, one way to accomplish the desired goal of reducing exhaled carbon dioxide is to place a carbon dioxide sequestering composition in a user's mouth, which is neither intrusive, uncomfortable, nor inconvenient—chewing gum, for example. Chewing gum compositions are already part of the global population's daily life because they are readily available, easily transported, and provide aesthetic or health benefits, such as fresh breath, cavity protection, and dietary supplement ingestion. Chewing gum is a convenient product which does not burden the user or require a break from daily routines. Moreover, chewing gum is placed in a user's mouth—the direct source of carbon dioxide exhalation emissions.

To further the goals of the green movement, there is a need for a method and composition which can reduce the amount of carbon dioxide emitted though exhalation by human beings. Because of its unobtrusiveness, convenience, and location of use, chewing gum and similar oral compositions are an ideal vehicle to implement a method which sequesters carbon dioxide in a user's breath, reducing carbon dioxide emissions from exhalation.

### SUMMARY OF THE INVENTION

The present invention provides a composition and a method for reducing the amount of carbon dioxide exhaled by its user. The composition comprises a delivery vehicle and a carbon dioxide sequestering agent. After the composition is delivered to a user's mouth, the carbon dioxide sequestering agent reacts with the carbon dioxide present in the user's breath to bind the gas in a form that is not reversible under bodily and common environmental conditions. As the agent reacts with the carbon dioxide in the user's breath, the carbon dioxide is consumed or reacted, thereby reducing the amount of carbon dioxide exhaled by the user. In certain embodiments, the delivery vehicle may be chewing gum. In other embodiments, the delivery vehicle may be a gas-permeable packet or taffy. In some embodiments, the carbon dioxide sequestering agent is calcium oxide, which in some embodiments is encapsulated before it is applied to, or incorporated in, the delivery vehicle. Additionally, the composition may further comprise a catalyst or buffering agent,

The method provided by the present invention comprises a user placing the composition comprising a carbon dioxide sequestering agent described above in their mouth, and allowing the agent to react with the carbon dioxide present in their breath, thereby reducing the quantity of carbon dioxide exhaled by the user.

### DETAILED DESCRIPTION

The present invention relates to a composition and method for reducing the amount of carbon dioxide exhaled by an individual. The following description is presented to enable one of ordinary skill in the art to make and use the invention and is provided in the context of a patent application and its requirements. Various modifications to the preferred embodiment will be readily apparent to those skilled in the art and the generic principles herein may be applied to other embodiments. Thus, the present invention is not intended to be limited to the embodiments shown but is to be accorded the widest scope consistent with the principles and features described herein.

The present invention is a method and composition for reducing the amount of carbon dioxide exhaled by a person by providing that person with a composition comprising a delivery vehicle and a carbon dioxide sequestering agent—the composition being intended to be placed in the mouth of the user. In the preferred embodiment, the delivery vehicle is a piece of chewing gum. In other embodiments, the delivery vehicle may take other forms, including but not limited to a tablet, a hard candy, a quick dissolving strip, a taffy, or a gas-permeable packet. Any medium which delivers the carbon dioxide sequestering agent into the user's mouth and allows for interaction between the agent and the carbon dioxide in the user's breath is a suitable form for use in the present invention. In the preferred embodiment, wherein the composition is a piece of chewing gum, as the user chews the gum the carbon dioxide sequestering agent is released or activated, which traps some of the carbon dioxide being exhaled by the user, thus reducing the amount of carbon dioxide expelled into the environment.

In the preferred embodiment, the chewing gum base is a water insoluble composition. In certain embodiments, the chewing gum formulation contains a gum base which may comprise elastomers, plasticizers, waxes, fats, oils, emulsifiers, fillers, texturizers, and the like. The chewing gum may further comprise flavorings and/or sweeteners. Various formulations and methods of production for the gum base are known in the art. For example, U.S. Pat. Nos. 3,821,417; 3,984,574; 4,041,179; 4,170,663; and 4,590,075 disclose various gum base formulations, materials, and methods of manufacturing. These patents are hereby fully incorporated by reference for all purposes.

In the preferred embodiment, the carbon dioxide sequestering agent comprises calcium oxide (CaO). At room temperature, calcium oxide will spontaneously react with carbon dioxide to form calcium carbonate (CaCO₃). This reaction is very slow. However, when calcium oxide is mixed with water (H₂O), the calcium oxide is converted to calcium hydroxide (Ca(OH)₂). Calcium hydroxide reacts with carbon dioxide to form calcium carbonate and water at an increased rate. The reaction is set forth below:

CaO+H₂O→Ca(OH)₂

Ca(OH)₂+CO₂→CaCO₃+H₂O

By providing calcium oxide in a chewing gum, as in the preferred embodiment, the calcium oxide reacts with the water in a user's saliva to form calcium hydroxide. The calcium hydroxide then reacts with the carbon dioxide present in the user's breath to form calcium carbonate and water. During the formation of calcium carbonate, the carbon dioxide from the user's breath is consumed, i.e. sequestered, and the amount of carbon dioxide exhaled by the user into the environment is reduced.

Forming the intermediate calcium hydroxide through the reaction of the calcium oxide with water in the user's saliva increases the rate of consumption of carbon dioxide from the user's breath. An increased reaction rate is desirable so that a sufficient amount of carbon dioxide may be consumed/sequestered during a gum chewing session. The reaction rate may further be increased by increasing the reaction temperature. Since the reaction of the carbon dioxide sequestering agent with carbon dioxide will take place in the user's mouth, the reaction rate will be increased close to or equal to body temperature, 98.6° F., which is well above room temperature. Indeed, the reaction temperature may be even higher than body temperature due to the extra. energy and heat generated by mastication of the gum. Specifically, the reaction may take place in the temperature range between 80° F. and 160° F., with the most common temperature range being between 98 and 105° F.

To further increase the rate of reaction, the chewing gum may additionally comprise a catalyst. For example, the concentration of water in a user's mouth during mastication may be increased to drive the formation of calcium hydroxide from calcium oxide. The concentration of water in the user's mouth may be increased through provision of an aqueous solution in the gum which is released during chewing. The aqueous solution may further comprise other ingredients, such as a sweetener, a colorant, a viscosity modifier, and/or a flavoring agent.

In addition to or in place of a catalyst, the chewing gum may comprise a buffering agent, which is released into the mouth during mastication to control the pH levels in the mouth during carbon dioxide sequestration. Acidic and basic buffering agents may be used. Examples of such buffering agents include, but are not limited to, citric acid, tartaric acid, malic acid, ascorbic acid, acid phosphate salts, calcium carbonate, magnesium oxide, sodium bicarbonate, 3-{[tris(hydroxymethyl)methyl]amino}propanesulfonic acid, N,N-bis(2-hydroxyethyl)glycine, tris(hydroxymethyl)methylamine, and N-tris(hydroxymethyl)methylglycine.

Calcium oxide is merely one example of a carbon dioxide sequestering agent. One of ordinary skill in the art will readily appreciate that any compound which reacts with carbon dioxide either directly or through an intermediate step—i.e., forming an intermediate compound—at near, equal to, or greater than body temperatures, may be a suitable carbon dioxide sequestering agent for use in other embodiments of the present invention. Examples of such agents include, but are not limited to, soda lime, barium hydroxide lime, Ca(OH)₂, Na₂CO₃, and Li₄SiO₄.

In the case of calcium oxide, the carbon dioxide sequestering agent provided in the composition is comprised of a single compound. In other embodiments, the carbon dioxide agent may be comprised of two or more compounds. In one such embodiment the two or more compounds may combine in the user's mouth to form a carbon dioxide sequestering agent which reacts with the carbon dioxide in the user's breath to reduce the total output of carbon dioxide through exhalation. The formed carbon dioxide sequestering agent may or may not react with water in the user's saliva and may or may not be converted into another compound before reacting with carbon dioxide in the user's breath. In another embodiment, the two or more compounds may not combine to form a carbon dioxide sequestering agent, but rather react with the carbon dioxide in the user's breath in their separate forms. In embodiments where the carbon dioxide sequestering agent is comprised of two or more compounds, the two or more compounds may be sequestered from each other, and prevented from coming into contact until, in the case of the preferred embodiment, the gum is chewed. In compositions comprising multi-compound carbon dioxide sequestering agents, the composition may further comprise a catalyst or accelerant.

In one embodiment, the delivery vehicle is spray coated or dusted with the carbon dioxide sequestering agent. In embodiments wherein the carbon dioxide sequestering agent comprises two or more compounds, the compounds may be sequestered by coating one side of the composition with one compound, and the other side of the composition with a different compound. In other embodiments, wherein the agent comprises two or more compounds, one half of the composition may be coated with one compound, and the other half coated with another compound. In another embodiment, the multiple compounds are imbedded throughout the composition, but are not mixed. Additionally, a catalyst or accelerant may be spray coated onto the composition with the agent and may also be spray coated in such a manner as to keep the catalyst or accelerant separate from the carbon dioxide sequestering agent.

In some embodiments the agent is not released from the composition. For example, in the preferred embodiment, the calcium oxide may not be released from the gum matrix, but rather remains bound in the matrix as the chemical reaction takes place. Similarly, any by-product from the reaction, such as calcium carbonate, may also remain bound to the gum matrix. This may be accomplished, for example, by imbedding the agent in the gum matrix or chemically-linking the agent to the gum matrix.

In some embodiments, the carbon dioxide sequestering agent may be present in a center filled composition whereby the agent is imbedded in the center of the composition, which can be in the same solid phase or dispersed in a liquid phase. In embodiments where the agent is comprised of more than one compound, one compound may be et bedded in the center f the composition white the other is spray coated on the surface of the composition.

The total weight of the carbon dioxide sequestering agent in a composition may vary to be less than 1 g, less than 700 mg, or between 1 and 300 mg.

Because carbon dioxide is naturally present in the atmosphere, certain precautions should be taken in the manufacturing of the composition and provision of the carbon dioxide sequestering agents. For example, in embodiments of the composition where the carbon dioxide sequestering agent is exposed to the atmosphere, as would be the case in embodiments where the delivery vehicle is spray coated with the agent, the composition should be manufactured under an inert gas, such as nitrogen or argon, and then immediately packaged in airtight or gas-impermeable packaging. In the preferred embodiment, the gum should be packaged in foil sealed plastic packaging rather than wrapping the gum in paper or foil.

Additionally, in the case where the carbon sequestering agent is calcium oxide, as well as in other embodiments where the agent reacts with water, the composition should be manufactured under low moisture conditions to prevent the early reaction of the agent.

If precautions are not taken during the manufacturing and packaging process, the carbon dioxide sequestering agent may react with atmospheric carbon dioxide rendering the composition incapable of sequestering carbon dioxide by the time it is delivered to the user's mouth. In one aspect, the goal of the present invention is to deliver as much of the carbon dioxide sequestering agent to the user's mouth in the neat, unreacted form. In certain embodiments, the percentage by weight of the carbon dioxide sequestering agent that is delivered to the user's mouth in unreacted form is greater than 98%, 95%, 90%, 80%, or 70%.

Beyond manufacturing and packaging techniques, choosing a carbon dioxide sequestering agent which reacts slowly at room temperatures furthers the goal of delivering as much unreacted agent as possible to a user's mouth. Additionally, the carbon dioxide sequestering agent may be microencapsulated to prevent it from reacting with atmospheric carbon dioxide prior to its delivery to the user's mouth. Microcapsules are small particles that consist of a core of active agent surrounded by a shell of coating material. The shell is designed to isolate the core material from the surrounding environment and in some instances, control the release of the agent. Preferably, the capsule does not react with the carbon dioxide sequestering agent and/or is gas-impermeable. By encapsulating the carbon dioxide sequestering agent in gas-impermeable capsules, the agent can be isolated from atmospheric carbon dioxide. After introduction into the user's mouth, the capsules are ruptured, releasing the agent into the user's mouth to react with carbon dioxide present in the user's breath.

Encapsulation ay be achieved through pan coating, fluidized coating, fluidized bed hot melt coating, and the like. Suitable hydrophilic encapsulation material may be gum arabic, gum karaya, gum tragacanth, guar gum, locust bean gum, xanthan gum, carrageenan, alginate salt, casein, dextran, pectin, agar, sorbitol, 2-hydroxyethyl starch, 2-aminoethyl starch, maltodextrin, amylodextrin, 2-hydroxyethyl cellulose, methyl cellulose, carboxymethyl cellulose salt, cellulose sulfate salt, polyvinylpyrrolidone, polyethylene oxide, polyvinyl alcohol/acetate, and the like. Suitable water-insoluble encapsulants include polyvinyl acetate, polyacrylamide, polyvinyl chloride, polystyrene, polyethylene, polyurethane, polymethacryiate, paraffin wax, carnauba wax, beeswax, stearyl alcohol, zein, shellac, edible fat, and the like.

Encapsulation is known in the art and is described in U.S. Pat. Nos. 4,673,577; 4,933,190; 5,139,794; and 5,629,035, for example, which are hereby fully incorporated for all purposes.

In one embodiment, calcium oxide is microencapsulated. Chewing gum is then coated and/or imbedded with the calcium oxide, either encapsulated or not. The chewing gum comprising the calcium oxide is then provided to a user. As the user chews the gum, the calcium oxide is either hydrated in the user's saliva or the capsules are ruptured by the mechanical forces generated by mastication, or in some embodiments dissolved by the user's saliva, or both, releasing the calcium oxide, The calcium oxide then reacts with the water in the user's saliva and carbon dioxide in the user's breath to form calcium carbonate.

In certain embodiments, the encapsulated carbon dioxide sequestering agent is in powder form. In other embodiments, the encapsulated agent is in a liquid form. If the agent is in a liquid form like a solution, suspension, or emulsion, the type of liquid in which the agent is dissolved will depend upon the characteristics of the agent. For example, if the carbon dioxide sequestering agent is calcium oxide, then the liquid in which the calcium oxide is dissolved should not contain water in order to prevent premature reaction of the calcium oxide.

In one embodiment, the composition may comprise one or more large capsules containing the carbon dioxide sequestering agent. For example, in embodiments wherein the delivery vehicle is chewing gum, the agent may be encapsulated in one large capsule and then embedded into the center of the chewing gum, In other embodiments, the composition comprises a plurality of microcapsules containing the agent. In some such embodiments, the microcapsules have an average size of between 20 and 400 microns, less than 20 microns, or have an average size of less than 1 micron.

In embodiments wherein the carbon dioxide sequestering agent comprises more than one compound and/or a catalyst or accelerant, the compounds and the catalyst or accelerant may all be encapsulated, or only a subset of the compounds and/or the catalyst or accelerant may be encapsulated. For example, one of the compounds may be encapsulated white the remaining compounds, catalyst, or accelerant, if present, are not encapsulated. In another embodiment, the one or more compounds may be encapsulated while the catalyst is not. Generally, encapsulating compounds when the carbon dioxide sequestering agent is comprised of one or more compounds isolates the compounds from each other and prevents premature reaction of the agent. In certain embodiments, however, encapsulating the compounds together may prove beneficial.

A method and composition for reducing the amount of carbon dioxide exhaled by an individual has been disclosed above. Although the present invention has been described in accordance with the embodiments shown, one of ordinary skill in the art will readily recognize that there could be variations to the embodiments shown and that those variations would be within the spirit and scope of the present invention.

For example, rather than chewing gum, the delivery vehicle may take the form of a gas-permeable packet which is placed in a user's mouth. Preferably, the packet is made out of a material that would not rupture if chewed by the user and is gas-permeable. In some embodiments, the packet is both gas-permeable and liquid-permeable allowing both carbon dioxide in the user's breath and the user's saliva to pass freely into the packet. The interior of the packet contains a carbon dioxide sequestering agent. In one embodiment the agent is calcium oxide. Preferably, the agent is not able to pass through the material of the packet, thus keeping the agent contained within the packet. As described above, the agent may be a unitary compound or comprise more than one compound. The packet may further contain a catalyst or accelerant, such as water, as well as buffering agents, colorants, sweeteners, and/or flavorants. In one embodiment, the components contained within the packet are not encapsulated. In other embodiments, some or all of the components are encapsulated in accordance with the principles previously described. When the user places the packet in their mouth, the capsules are ruptured either through mechanical forces or dissolved by the user's saliva, releasing the carbon dioxide sequestering agent within the packet. The agent then reacts with the carbon dioxide in the user's breath which passes freely through the packet material, reducing the amount of carbon dioxide exhaled by the user. By using a packet as the delivery vehicle, any by-product of the reaction is contained in the packet and is discarded when the user throws away the packet after use.

Accordingly, many modifications may be made by one of ordinary skill in the art without departing from the spirit and scope of the appended claims.

What is claimed is:

1. A composition which is placed in a user's mouth comprising:
a delivery vehicle; and
a carbon dioxide sequestering agent which reacts with carbon dioxide present in the user's breath reducing the amount of carbon dioxide exhaled by the user.

2. The composition of claim 1 wherein the delivery vehicle is a chewing gum, a tablet, a hard candy, a quick dissolving strip, a taffy, or a gas-permeable packet.

3. The composition of claim 1 wherein the delivery vehicle is a chewing gum.

4. The composition of claim l wherein the delivery vehicle is a gas permeable packet.

5. The composition of claim 1 wherein the carbon dioxide sequestering agent is comprised of a single compound.

6. The composition of claim 1 wherein the carbon dioxide sequestering agent is comprised of two or more compounds.

7. The composition of claim 5 wherein the carbon dioxide sequestering agent forms an intermediate compound before reacting with carbon dioxide.

8. The composition of claim 1 wherein the carbon dioxide sequestering agent is calcium oxide.

9. The composition of claim 1 wherein the carbon dioxide sequestering agent is selected from soda lime, barium hydroxide lime, Ca(OH)₂, Na₂CO₃, and Li₄SiO₄.

10. The composition of claim 1 further comprising a catalyst.

11. The composition of claim 1 further comprising a buffering agent.

12. The composition of claim 1 wherein the carbon dioxide sequestering agent is encapsulated.

13. A composition which is placed in the user's mouth comprising:
chewing gum; and
calcium oxide.

14. The composition of claim 13 wherein the calcium oxide is spray coated on the chewing gum.

15. The composition of claim 13 wherein the calcium oxide is encapsulated.

16. The composition of claim 15 wherein the encapsulated calcium oxide is spray coated on the chewing gum.

17. The composition of claim 16 further comprising a catalyst.

18. The composition of claim 16 further comprising a buffering agent.

19. The composition of claim 13 wherein 95% or more of the calcium oxide is in an unreacted form at the time the composition is placed in the user's mouth.

20. A method for reducing the amount of carbon dioxide exhaled by a user comprising:
providing the user with a composition comprising a delivery vehicle and a carbon dioxide sequestering agent;
placing the composition into the user's mouth; and
allowing the carbon dioxide sequestering agent to react with the carbon dioxide in the user's breath, thereby reducing the amount of carbon dioxide exhaled by the user.

21. The method of claim 20 wherein the delivery vehicle is chewing gum.

22. The method of claim 21 wherein the carbon dioxide sequestering agent is calcium oxide.

23. The method of claim 21 wherein the carbon dioxide sequestering agent is selected from soda lime, barium hydroxide lime, Ca(OH)₂, Na₂CO₃, and Li₄SiO₄.
